(19) United States
(12) Patent Application Publication
(10) Pub. No.: US 2023/0293016 A1
(43) Pub. Date: Sep. 21, 2023

(54) METHOD AND DEVICE FOR TESTING FOR FOOT ULCER

(71) Applicant: Ki Wan PARK, Seoul (KR)

(72) Inventor: Ki Wan PARK, Seoul (KR)

(21) Appl. No.: 18/013,298

(22) PCT Filed: Apr. 23, 2021
(86) PCT No.: PCT/KR2021/005142

§ 371 Date: Dec. 28, 2022

(30) Foreign Application Priority Data
Jul. 2, 2020 (KR) 10-2020-0081403

(51) Int. Cl. A61B 5/00

(52) U.S. Cl. CPC A61B 5/0051; A61B 5/445

(57) ABSTRACT

The present invention relates to a method and a device for testing for a foot ulcer and, particularly, to a method and a device for testing for a foot ulcer, wherein the method comprises the steps of: inputting an initial value into a terminal; operating, on the basis of the initial value, at least one of multiple stimulation devices provided in a body having a shape corresponding to the shape of the bottom of the foot; inputting a checkup value into the terminal; determining whether the checkup value corresponds to the operated stimulation device; and outputting the determination value to the terminal, and the device comprises: a body having a shape corresponding to the shape of the bottom of the foot; multiple stimulation devices provided in the body and spaced apart from each other; a communication device connected to the multiple stimulation devices so as to operate a random stimulation device among the stimulation devices; and a terminal comprising a signal input unit, a control unit for randomly selecting the random stimulation device at the time of inputting of a signal and transmitting the signal to the communication device to derive a value, and an output unit for outputting the value derived by the control unit, whereby due to the configuration, a user can test, by himself/herself, whether a foot ulcer has developed.

### TECHNICAL FIELD

The present disclosure relates to a method and device for examining a foot ulcer, and more particularly to a method and device for examining a foot ulcer capable of checking, by a user alone, whether the foot ulcer has developed.

### BACKGROUND ART

When an inflammation has developed on the foot, it means that you cannot properly feel a touch in any part of the foot. Therefore, in order to examine a condition of the foot, there is a method of checking whether you properly feel a stimulation by stimulating a part of the foot which is particularly inflamed or has a tactile nerve.

In general, as a method of examining a diabetic foot ulcer, there is a ‘10 gram monofilament test’ method of pressing an arbitrary point of the sole of the foot to such an extent that a monofilament is bent and checking whether a user notices a press and the arbitrary point.

In this method, an examiner presses several points on the sole of the foot that are suspected of being inflamed one by one while preventing the user from seeing it and makes the user guess which part of the sole of the foot is pressed to check whether the user properly feels the stimulation.

As another method, an examination may also be performed by lightly applying a pressure to the sole of the foot with a finger. However, it is difficult for the user to examine by himself/herself because he or she knows that the pressure is applied even though he/she cannot feel a touch when he/she sees the pressure applied. Hence, there is also a method of examining by applying the pressure to various parts of the sole of the foot in a state where the user is not able to see in such a way that the user is turned over so that the user does not know that the pressure is applied.

In the above methods, the user should not look at the sole of his/her foot during the examination. Therefore, the user cannot perform the examination by himself/herself, and an examiner is required.

As a device related to the above examination, a temperature measurement device and a temperature measurement method for early diagnosing a pre-ulcer state of diabetes are disclosed in Korean Patent Application Publication No. 10-2001-0074696 (patent document 1).

The patent document 1 includes a controller, a temperature sensing means, a contact sensing means, a display, etc., and diagnoses a pre-ulcer state of diabetes based on a local increase of a skin temperature being one of early signs of tissue damage or inflammation.

However, in the patent document 1, a temperature of the sole of the foot may be measured differently from an actual condition of the user depending on the individual user's physical condition or an external environment at the time of measurement, reducing reliability of a measurement result. In addition, if the user has to perform an examination by himself/herself, it is difficult to examine an exact position because the user needs to move the temperature measurement device to different points on the sole of the foot each time the measurement is performed.

### PRIOR ART DOCUMENT

### Patent Document

(Patent Document 1) Korean Patent Application Publication No. 10-2001-0074696 (Aug. 9, 2001)

### DISCLOSURE

### Technical Problem

An object of the present disclosure is to address the above-described and other problems. Another object of the present disclosure is to provide a method, device, and system for examining a foot ulcer capable of checking, by a user alone, whether the foot ulcer has developed.

Another object of the present disclosure is to provide a method and device for examining a foot ulcer capable of easily checking whether the foot ulcer has developed.

Another object of the present disclosure is to provide a method and device for examining a foot ulcer with improved examination reliability.

### Technical Solution

In order to achieve the above-described and other objects, in one aspect of the present disclosure, there is provided a method (S100) of examining a foot ulcer comprising a step (S110) of inputting a start value to a terminal including a controller; a step (S120) of randomly selecting, by the controller, one stimulation device among a plurality of stimulation devices connected to a communication device included in each of a left main body and a right main body of a shape corresponding to a shape of a sole of a foot; a step (S130) of transmitting, by the controller, a signal to the communication device connected to the selected stimulation device; a step (S140) of operating the selected stimulation device; a step (S150) of inputting a check value to the terminal; a step (S160) of deriving, by the terminal, a determination value about whether the check value matches the operated stimulation device; and a step (S170) of outputting the determination value to the terminal.

According to one aspect of the present disclosure, after the random selection step (S120) to the determination value deriving step (S160) are repeated a predetermined number of times, the determination value output step (S170) may be performed. In the determination value output step (S170), the determination values derived by the predetermined number of times may be synthesized and output.

According to one aspect of the present disclosure, in the operation step (S140), the stimulation devices may generate a vibration, and the stimulation devices may be operated at the same intensity of stimulation.

In another aspect of the present disclosure, there is provided a method (S200) of examining a foot ulcer comprising a step (S210) of inputting a start value to a terminal; a step (S215) of transmitting the start value to a server; a step (S220) of randomly selecting, by the server, one stimulation device among a plurality of stimulation devices included in a main body of a shape corresponding to a shape of a sole of a foot; a step (S230) of transmitting, by the server, a signal operating the selected stimulation device to a communication device connected to the plurality of stimulation devices; a step (S240) of operating, by the communication device, the selected stimulation device; a step (S250) of inputting a check value to the terminal; a step (S255) of transmitting the check value to the server; a step (S260) of deriving, by the server, a determination value about whether the check value matches the operated stimulation device; and a step (S270) of transmitting, by the server, the determination value to the terminal and outputting the determination value to the terminal.

In another aspect of the present disclosure, there is provided a foot ulcer examination device comprising a main body of a shape corresponding to a shape of a sole of a foot; a plurality of stimulation devices included in the main body and spaced apart from each other; and a communication device connected to the plurality of stimulation devices and a terminal randomly selecting the stimulation device to be operated, the communication device being configured to operate the stimulation device selected by the terminal.

In another aspect of the present disclosure, there is provided a foot ulcer examination system comprising a main body of a shape corresponding to a shape of a sole of a foot; a plurality of stimulation devices included in the main body and spaced apart from each other; a communication device connected to the plurality of stimulation devices and configured to operate the stimulation device; and a terminal including an input unit configured to obtain an input value, a controller connected to the input unit and the communication device and configured to perform a calculation on the input value and randomly select the stimulation device to be operated, and an output unit configured to output an output value processed by the controller.

### Advantageous Effects

According to a method, device, and system for examining a foot ulcer according to an embodiment of the present disclosure, since a terminal can operate and check a stimulation device that applies a stimulation to the sole of the foot, a user can examine a sign of foot ulcer by himself or herself and early find the sign of foot ulcer.

According to a method, device, and system for examining a foot ulcer according to an embodiment of the present disclosure, since the same position on the sole of the foot is examined at the same intensity of stimulation, examination reliability of the foot ulcer can be improved.

### DESCRIPTION OF DRAWINGS

FIG. 1a illustrates left and right stimulation assemblies according to a first embodiment of the present disclosure.

FIG. 1b illustrates left and right stimulation assemblies according to a third embodiment of the present disclosure.

FIG. 2a illustrates a configuration of a foot ulcer examination system according to first and third embodiments of the present disclosure.

FIG. 2b illustrates a configuration of a foot ulcer examination system according to a second embodiment of the present disclosure.

FIG. 3 illustrates an example of a method of using a foot ulcer examination system.

FIG. 4a is a flow chart illustrating a foot ulcer examination method according to a first embodiment of the present disclosure.

FIG. 4b is a flow chart illustrating a foot ulcer examination method according to a second embodiment of the present disclosure.

### MODE FOR INVENTION

In embodiments of the present disclosure to be described below, reference for the same structure and components as those of a related art is made to the related art, and a detailed description thereof is omitted.

The technical terms disclosed herein are used to merely refer to a specific embodiment and does not intend to limit the present disclosure. A singular expression used in embodiments can include a plural expression as long as it does not have an apparently different meaning in context. In the present disclosure, terms “include” and “comprise” should be understood to be intended to designate that illustrated features, areas, numbers, steps, operations, components, parts and/or combinations thereof are present and not to preclude the existence of one or more different features, areas, numbers, steps, operations, components, parts and/or combinations thereof, or the possibility of the addition thereof.

When any component is described as “being connected” or “being coupled” to other component, this should be understood to mean that another component may exist between them, although any component may be directly connected or coupled to the other component.

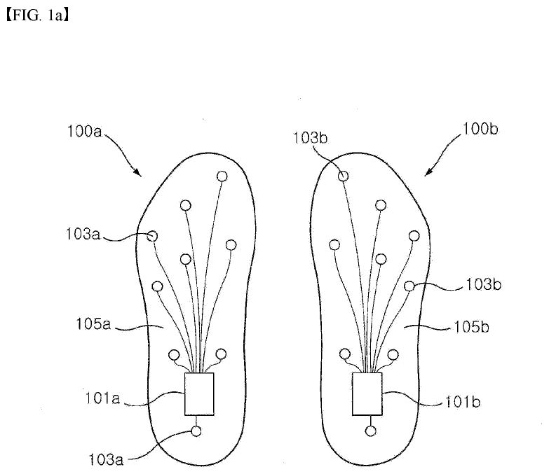

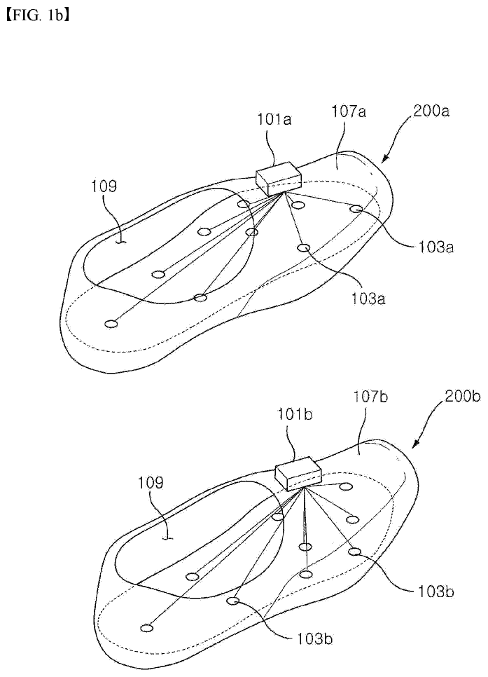

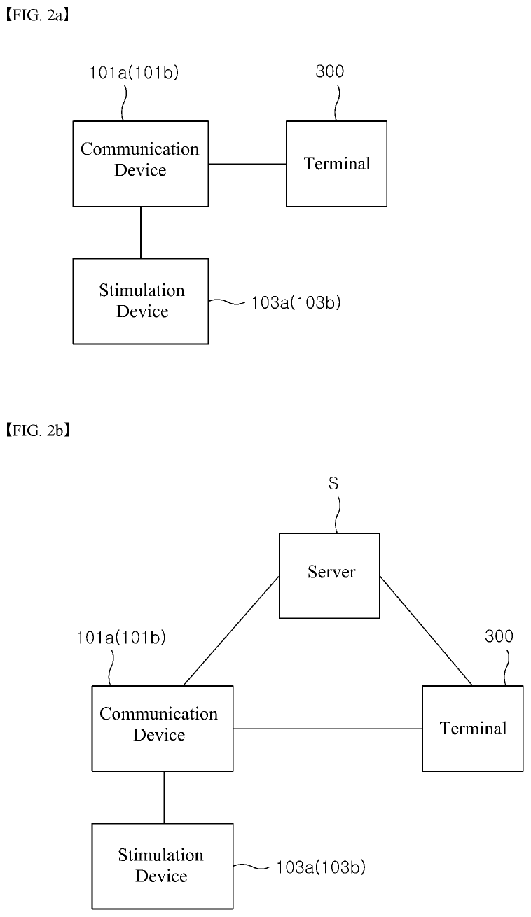

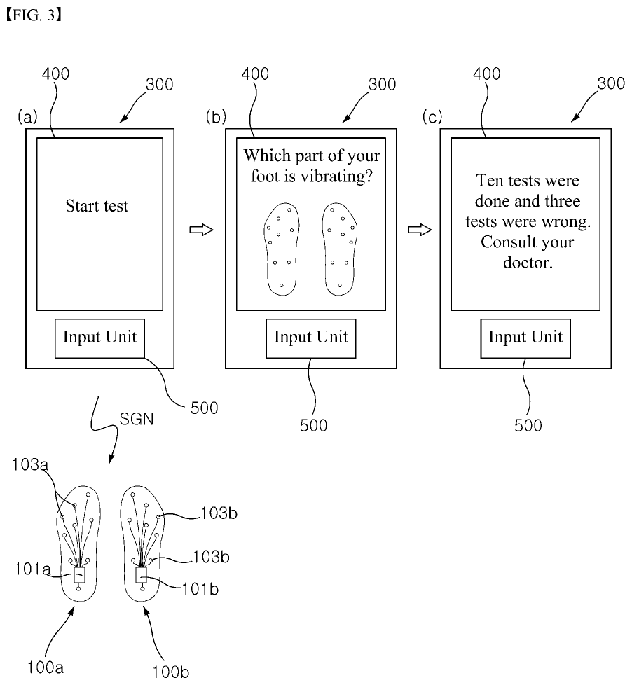

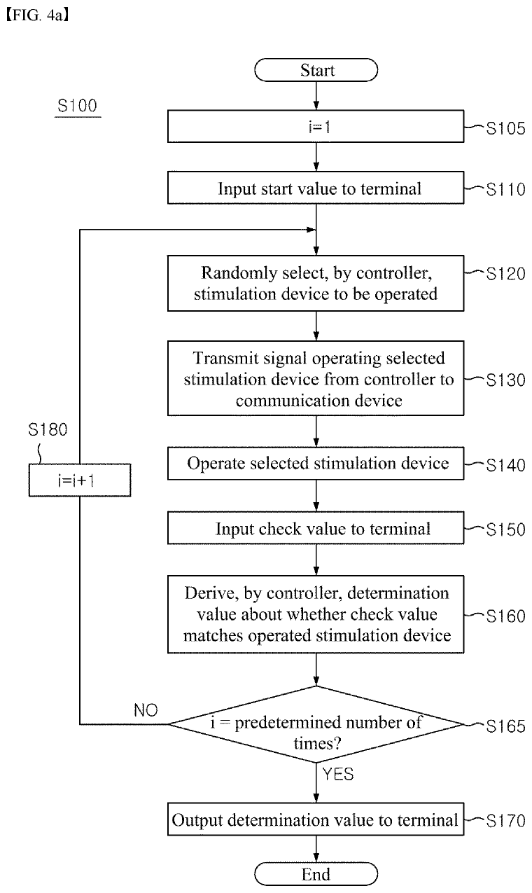

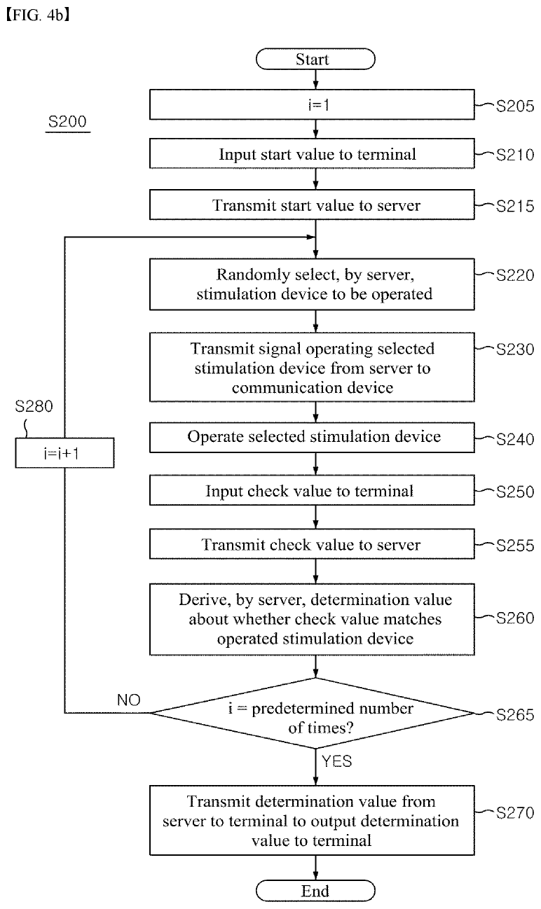

FIG. 1a is a plan view of left and right stimulation assemblies 100a and 100b according to a first embodiment of the present disclosure. FIG. 1b is a perspective view of left and right stimulation assemblies 200a and 200b according to a third embodiment of the present disclosure. FIG. 2a illustrates a configuration of a foot ulcer examination system according to the first and third embodiments of the present disclosure. FIG. 2b illustrates a configuration of a foot ulcer examination system according to a second embodiments of the present disclosure. FIG. 3 illustrates an example of a method of using a foot ulcer examination system. FIG. 4a is a flow chart illustrating a foot ulcer examination method according to the first embodiment of the present disclosure. FIG. 4b is a flow chart illustrating a foot ulcer examination method according to the second embodiment of the present disclosure.

The present disclosure illustrates that the number of stimulation devices 103a and 103b for each foot is nine in total, but is not limited thereto.

Referring to FIGS. 1a and 2a, a foot ulcer examination device according to the first embodiment of the present disclosure may include main bodies 105a and 105b having a shape corresponding to a sole shape; a plurality of stimulation devices 103a and 103b that are included in the main bodies 105a and 105b and are disposed to be spaced apart from each other; and communication devices 101a and 101b that are connected to a terminal 300 randomly selecting the stimulation device to be operated, are respectively connected to the plurality of stimulation devices 103a and 103b, and operate the stimulation devices 103a and 103b selected by the terminal 300 among the plurality of stimulation devices 103a and 103b.

Referring to FIGS. 1a and 2a, a foot ulcer examination system according to the first embodiment of the present disclosure may include the foot ulcer examination device and the terminal 300 including an input unit 500 to which an input value is input, a controller (not shown) that is connected to the input unit 500 and the communication devices 101a and 101b to perform a calculation on the input value and randomly selects the stimulation devices 103a and 130b to be operated, and an output unit 400 outputting a value calculated by the controller (not shown).

According to the present disclosure including the above-described configuration, a user can easily check the development of ulcer on various parts of the sole of the foot by himself/herself, and can find signs of foot ulcer at an early stage.

Hereinafter, the main bodies 105a and 105b and the stimulation devices 103a and 103b are collectively referred to as stimulation assemblies 100a and 100b. For example, the stimulation assemblies 100a and 100b may mean at least one of the main bodies 105a and 105b and the stimulation devices 103a and 103b. For example, the stimulation assemblies 100a and 100b may include the main bodies 105a and 105b and the stimulation devices 103a and 103b.

As illustrated in FIG. 1a, the stimulation assemblies 100a and 100b may be provided with stimulation devices for a left foot and a right foot so that both feet can be examined at the same time. That is, the stimulation assemblies 100a and 100b may include a left stimulation assembly 100a having a left main body 105a and a left communication device 101a and a right stimulation assembly 100b having a right main body 105b and a right communication device 101b. The left main body 105a and the right main body 105b may be included in the main bodies 105a and 105b. The left communication device 101a and the right communication device 101b may be included in the communication devices 101a and 101b.

The main bodies 105a and 105b according to the first embodiment of the present disclosure are in the form of an insole.

Upper surfaces of the main bodies 105a and 105b are in contact with the sole of the user's foot.

The stimulation devices 103a and 103b and the communication devices 101a and 101b are positioned inside the main bodies 105a and 105b and are not exposed to the outside. Thus, the stimulation devices 103a and 103b is waterproof and durable.

The communication devices 101a and 101b may be easily connected to the stimulation devices 103a and 103b. It is preferable that the communication devices 101a and 101b are positioned between the plurality of stimulation devices 103a and 103b for the purpose of durability.

As illustrated in FIG. 1a, the stimulation devices 103a and 103b may be positioned to examine a big toe, a middle toe, and a little toe, i.e., three positions and the anterior, middle, and posterior of the sole, i.e., six positions. Thus, the present disclosure can examine the development of ulcer on various parts of the sole of the foot.

The connection method of the communication devices 101a and 101b and the stimulation devices 103a and 103b illustrated in FIGS. 1a and 1b is merely an example and is not limited to FIGS. 1a and 1b.

When a start value to be described later, which is a value for starting the examination, is input to the input unit 500, the controller (not shown) may perform an operation of randomly selecting the stimulation devices 103a and 103b to be operated. The controller (not shown) may also perform an operation of determining whether a check value to be described later input to the input unit 500 matches the selected stimulation devices 103a and 103b.

As illustrated in FIG. 2b, a foot ulcer examination system according to a second embodiment of the present disclosure may include a server S serving as the controller (not shown), a foot ulcer examination device, and a terminal 300. Thus, the terminal 300 according to the second embodiment may not require the controller (not shown).

The server S may be wirelessly connected to the terminal 300 and communication devices 101a and 101b.

A value input through an input unit 500 of the terminal 300 may be transmitted to the server S.

Stimulation devices 103a and 103b to be operated may be randomly selected from the server S.

The server S may perform an operation of determining whether a check value matches the stimulation devices 103a and 103b, that are randomly selected and operated, to derive a determination value and may send the determination value to the terminal 300.

Configurations and effects not described herein refer to the configurations and effects of the first embodiment.

Referring to FIG. 1b, in a foot ulcer examination system according to a third embodiment of the present disclosure, main bodies 107a and 107b have a different shape from those of the first embodiment.

Specifically, the main bodies 107a and 107b of stimulation assemblies 200a and 200b may be in the form of socks or shoes and may have a shape corresponding to a shape of the foot.

The main bodies 107a and 107b may include an accommodating portion 109 with an open upper part so that a user's feet can be accommodated therein.

The soles and tops of the feet of the user may contact inner surfaces of the main bodies 107a and 107b. Therefore, since the user's feet do not depart from or move from the main bodies 107a and 107b, the same position at the user's feet can be examined each time the examination is performed, and reliability of examination result can be improved.

As illustrated in FIG. 1b, communication devices 101a and 101b according to the third embodiment of the present disclosure may be positioned on the upper part of the main bodies 107a and 107b, i.e., on the instep.

Configurations and effects not described herein refer to the configurations and effects of the first embodiment.

Although not illustrated, when the main bodies 107a and 107b are in the form of socks or shoes as in the third embodiment, the communication devices 101a and 101b may be positioned on the user's ankle using a strap, etc.

A foot ulcer examination method according to the first embodiment of the present disclosure comprises a step S110 of inputting a start value to a terminal 300 including a controller (not shown); a step S120 of randomly selecting, by the controller (not shown), one stimulation device (103a, 103b) among a plurality of stimulation devices (103a and 103b) connected to a communication device (101a and 101b) included in each of a left main body (105a, 105b) and a right main body (107a, 107b) of a shape corresponding to a shape of sole of foot; steps S130 and S140 of transmitting, by the controller (not shown), a signal SGN to the communication device (101a and 101b) connected to the selected stimulation device (103a and 103b) to operate the selected stimulation device (103a and 103b); a step S150 of inputting a check value to the terminal 300; a step S160 of deriving, by the terminal 300, a determination value about whether the check value matches the operated stimulation device (103a and 103b); and a step S170 of outputting the determination value to the terminal 300.

According to the above description, in the foot ulcer examination method, the user has to guess whether the stimulation devices 103a and 103b are operating, by a sense without looking with eyes. Thus, it is possible to examine whether there is a part where the user does not well feel the vibration by himself/herself, that is, whether the foot ulcer has developed.

The output step S170 may be performed after the random selection step S120 to the determination value derivation step S160 are repeated a predetermined number of times.

The output step S170 is a step of synthesizing the determination values derived by the predetermined number of times to output the synthesized value to the output unit 400.

As an example of synthesizing and outputting the determination values derived by the predetermined number of times in the output step S170, as illustrated in FIG. 3, the sentence such as “Ten tests were done and three tests were wrong. Consult your doctor” may be output to the output unit 400 of the terminal 300. Through this, it can help prevent the foot ulcer by providing the user with the number of inconsistencies and recommending that the user consults with a doctor in case of inconsistency.

When the operation step S140 is repeated a predetermined number of times, it is preferable that the previously unoperated stimulation devices 103a and 103b among the plurality of stimulation devices 103a and 103b are operated each time. Thus, the examination time can be reduced by not examining again a position once examined.

The operation step S140 may be a step in which the stimulation devices 103a and 103b generate vibration, and in this case, the stimulation devices 103a and 103b are operated at the same intensity of stimulation. Thus, the present disclosure can improve the reliability of the examination since the development of the ulcer is checked at the same intensity of vibration while the examination is repeated a predetermined number of times.

The determination value derivation step S160 is a step in which the controller (not shown) derives a determination value through an operation of determining whether the check value matches the operated stimulation devices 103a and 103b.

A foot ulcer examination method S100 according to the first embodiment of the present disclosure is described in detail below with reference to FIGS. 3 and 4a.

The foot ulcer examination method S100 according to the first embodiment of the present disclosure may comprise a step S105 of initializing the number of examinations. In the step S105, the number of examinations (i) may be set to 1.

The foot ulcer examination method S100 according to the first embodiment of the present disclosure may comprise a step S110 of inputting a start value to the terminal 300. For example, the input unit 500 of the terminal 300 may obtain the start value. For example, the start value input to the input unit 500 may indicate an input value. For example, the start value input to the input unit 500 is a setting value for starting the foot ulcer examination method S100 according to the first embodiment of the present disclosure and may be referred to as a “first input value”. That is, in a state illustrated in FIG. 3(a), the input value starting the foot ulcer examination by the user may be input to the input unit 500 of the terminal 300, and the input value may be transmitted to the controller (not shown), in S110.

The foot ulcer examination method S100 according to the first embodiment of the present disclosure may comprise a step S120 of randomly selecting stimulation devices 103a and 103b to be operated among a plurality of stimulation devices 103a and 103b. In the step S120, the controller (not shown) of the terminal 300 may randomly select stimulation devices 103a and 103b to be operated among the plurality of stimulation devices 103a and 103b. For example, the controller (not shown) may select one stimulation device (103a, 103b) from each of a left main body (105a, 107a) and a right main body (105b, 107b).

The foot ulcer examination method S100 according to the first embodiment of the present disclosure may comprise a step S130 of transmitting, by the terminal 300, a signal SGN for operating the selected stimulation devices 103a and 103b to the stimulation assemblies 100a and 100b. In the step S130, the terminal 300 may transmit the signal SGN to the stimulation assemblies 100a and 100b. The signal SGN may include information on the input value. For example, in the step S130, the terminal 300 may transmit the signal SGN to the communication devices 101a and 101b of the stimulation assemblies 100a and 100b.

The foot ulcer examination method S100 according to the first embodiment of the present disclosure may comprise a step S140 of operating the stimulation devices 103a and 103b. For example, in the step S140, the communication devices 101a and 101b may operate the stimulation devices 103a and 103b in response to the signal SGN. The step S140 may be repeated a predetermined number of times during the examination. For example, each time the step S140 is performed, the stimulation devices 103a and 103b may provide a stimulation of the same intensity of vibration.

The foot ulcer examination method S100 according to the first embodiment of the present disclosure may comprise a step S150 of inputting a check value to the terminal 300. In the step S150, the user may input the check value to the input unit 500 of the terminal 300. The check value may include information about a part where the user thinks he/she felt the stimulation. In the step S150, as illustrated in FIG. 3(b), positions of the stimulation devices 103a and 103b of left and right assemblies 100a, 100b, 200a, and 200b may be displayed on the input unit 500 of the terminal 300 so that the user easily inputs the check value. The check value may be referred to as a “second input value”. The selected stimulation devices 103a and 103b may continue to operate until the check value is input to the terminal 300.

The foot ulcer examination method S100 according to the first embodiment of the present disclosure may comprise a step S160 of deriving a determination value. The step S160 may be performed by the controller (not shown) of the terminal 300. The determination value may include information about whether the check value matches the operated stimulation devices 103a and 103b. The foot ulcer examination method S100 according to the first embodiment of the present disclosure may comprise a step S165 of comparing the number of examinations (i) with a predetermined number of times. In the foot ulcer examination method S100 according to the first embodiment of the present disclosure, the selection step S120 to the determination value deriving step S160 may be repeated the predetermined number of times, so that all the stimulation devices 103a and 103b are operated once. In the step S165, the controller (not shown) may decide whether the current number of examinations (i) equals the predetermined number of times.

The foot ulcer examination method S100 according to the first embodiment of the present disclosure may comprise a step S180 of increasing the number of examinations. For example, when the number of examinations (i) is not the same as the predetermined number of times, in the step S180, the number of examinations (i) may be increased by 1, and the step S120 of selecting the stimulation devices 103a and 103b may be performed. The step S120 of selecting the stimulation devices 103a and 103b to the step S160 of deriving the determination value may be repeated the predetermined number of times. The predetermined number of times may be a natural number.

The foot ulcer examination method S100 according to the first embodiment of the present disclosure may comprise a step S170 of outputting the determination value to the terminal 300. When the number of examinations (i) is the same as the predetermined number of times, the step S170 may be performed. In the step S170, the determination values derived over the predetermined number of times may be output to the output unit 400 of the terminal 300. For example, as illustrated in FIG. 3(c), since several examinations of the total number of examinations are wrong, a message that recommends consultation with a doctor may be displayed on the output unit 400. As another example, a warning may be output to the terminal 300, or a button that performs an additional examination may be displayed on the output unit 400. As another example, in the step S170, when the terminal 300 obtains an input for the output of the determination value, the determination value may be output to the output unit 400.

A foot ulcer examination method S200 according to the second embodiment of the present disclosure relates to a method of utilizing a foot ulcer examination system according to the second embodiment including the server S instead of the controller (not shown) in the first embodiment.

Specifically, the foot ulcer examination method S200 according to the second embodiment of the present disclosure may comprise a step S210 of inputting a start value to a terminal 300; a step S215 of transmitting the start value to a server S; a step S220 of randomly selecting, by the server S, one stimulation device (103a, 103b) among a plurality of stimulation devices (103a and 103b) included in main bodies 105a, 105b, 107a, and 107b of a shape corresponding to a shape of soles of feet; a step S230 of transmitting, by the server S, a signal SGN for operating the selected stimulation devices 103a and 103b to communication devices 101a and 101b connected to the plurality of stimulation devices 103a and 103b; a step S240 of operating, by the communication devices 101a and 101b, the selected stimulation devices 103a and 103b; a step S250 of inputting a check value to the terminal 300; a step S255 of transmitting the check value to the server S; a step S260 of deriving, by the server S, a determination value about whether the check value matches the operated stimulation devices 103a and 103b; and a step S270 of transmitting, by the server S, the determination value to the terminal 300 to output the determination value to the terminal 300.

Since the foot ulcer examination method S200 according to the second embodiment of the present disclosure uses the server S, a load that may be generated in the terminal 300 can be reduced, compared to the first embodiment.

The foot ulcer examination method S200 according to the second embodiment of the present disclosure is described in detail below with reference to FIGS. 2(b), 3 and 4b.

The foot ulcer examination method S200 according to the second embodiment of the present disclosure may comprise a step S205 of initializing the number of examinations. In the step S205, the number of examinations (i) may be set to 1.

The foot ulcer examination method S200 according to the second embodiment of the present disclosure may comprise the step S210 of inputting the start value to the terminal 300. For example, the input unit 500 of the terminal 300 may obtain the start value. For example, the start value input to the input unit 500 may indicate an input value. For example, the start value input to the input unit 500 is a setting value for starting the foot ulcer examination method S200 according to the second embodiment of the present disclosure and may be referred to as a “first input value”. That is, in a state illustrated in FIG. 3(a), the input value for the foot ulcer examination by the user may be input to the input unit 500 of the terminal 300, and the input value may be transmitted from the terminal 300 to the server S, in S215.

The foot ulcer examination method S200 according to the second embodiment of the present disclosure may comprise the step S220 of randomly selecting stimulation devices 103a and 103b to be operated among the plurality of stimulation devices 103a and 103b. Subsequently, in the step S220, the server S may randomly select stimulation devices 103a and 103b to be operated among the plurality of stimulation devices 103a and 103b. For example, the server S may select one stimulation device (103a, 103b) from each of a left main body (105a, 107a) and a right main body (105b, 107b).

The foot ulcer examination method S200 according to the second embodiment of the present disclosure may comprise the step S230 of transmitting, by the server S, the signal SGN for operating the selected stimulation devices 103a and 103b to the stimulation assemblies 100a and 100b. In the step S230, the server S may transmit the signal SGN to the stimulation assemblies 100a and 100b. The signal SGN may include information on the input value. For example, in the step S230, the server S may transmit the signal SGN to the communication devices 101a and 101b of the stimulation assemblies 100a and 100b. The foot ulcer examination method S200 according to the second embodiment of the present disclosure may comprise the step S240 of operating the stimulation devices 103a and 103b. For example, in the step S240, the communication devices 101a and 101b may operate the stimulation devices 103a and 103b in response to the signal SGN. The step S240 may be repeated a predetermined number of times during the examination. For example, each time the step S240 is performed, the stimulation devices 103a and 103b may provide a stimulation of the same intensity of vibration.

The foot ulcer examination method S200 according to the second embodiment of the present disclosure may comprise the step S250 of inputting the check value to the terminal 300. In the step S250, the user may input the check value to the input unit 500 of the terminal 300. The check value may include information about a part where the user thinks he/she felt the stimulation. In the step S250, as illustrated in FIG. 3(b), positions of the stimulation devices 103a and 103b of the left and right assemblies 100a, 100b, 200a, and 200b may be displayed on the input unit 500 of the terminal 300 so that the user easily inputs the check value. The check value may be referred to as a “second input value”. The selected stimulation devices 103a and 103b may continue to operate until the check value is input to the terminal 300.

The foot ulcer examination method S200 according to the second embodiment of the present disclosure may comprise the step S255 of transmitting the check value input to the terminal 300 to the server S.

The foot ulcer examination method S200 according to the second embodiment of the present disclosure may comprise the step S260 of deriving the determination value. The step S260 may be performed by the server S. The determination value may include information about whether the check value matches the operated stimulation devices 103a and 103b.

The foot ulcer examination method S200 according to the second embodiment of the present disclosure may comprise a step S265 of comparing the number of examinations (i) with a predetermined number of times. In the foot ulcer examination method S200 according to the second embodiment of the present disclosure, from the selection step S220 to the determination value deriving step S260 may be repeated a predetermined number of times, so that all the stimulation devices 103a and 103b are operated once. In the step S265, the server S may decide whether a current number of examinations (i) equals the predetermined number of times.

The foot ulcer examination method S200 according to the second embodiment of the present disclosure may comprise a step S280 of increasing the number of examinations. For example, when the number of examinations (i) is not the same as the predetermined number of times, in the step S280, the number of examinations (i) may be increased by 1, and the step S220 of selecting the stimulation devices 103a and 103b may be performed. The step S220 of selecting the stimulation devices 103a and 103b to the step S260 of deriving the determination value may be repeated the predetermined number of times. The predetermined number of times may be a natural number.

The foot ulcer examination method S200 according to the second embodiment of the present disclosure may comprise a step S270 of transmitting the determination value to the terminal 300. When the number of examinations (i) is the same as the predetermined number of times, the step S270 may be performed. In the step S270, the determination values derived over the predetermined number of times may be transmitted from the server S to the terminal 300 and displayed on the output unit 400. For example, as illustrated in FIG. 3(c), since several examinations of the total number of examinations are wrong, a message that recommends consultation with a doctor may be displayed on the output unit 400. As another example, a warning may be output to the terminal 300, or a button that performs an additional examination may be displayed on the output unit 400.

As another example, in the step S270, when the terminal 300 obtains an input for the output of the determination value, the determination value may be output to the output unit 400.

Configurations, steps and effects other than those described above with respect to the foot ulcer examination method S200 according to the second embodiment of the present disclosure may refer to the foot ulcer examination method S100 according to the first embodiment of the present disclosure.

Unlike the embodiments described above, the selection step (S120, S220) to the determination value deriving step (S160, S260) may be repeated so that at least two stimulation devices 103a and 103b are operated without having to operate all the stimulation devices 103a and 103b. In the embodiments described above, a mobile phone, a smart phone, etc. may be applied as the terminal 300.

The input unit 500 of the terminal 300 may include a button 500 or a touch screen. The output unit 400 of the terminal 300 may include a touch screen or a display. The communication devices 101a and 101b may be wireless communication devices 101a and 101b, and may be operated in a Bluetooth manner, for example.

In the embodiments described above, the stimulation devices 103a and 103b may generate vibration. The stimulation devices 103a and 103b may include at least one of a vibration sensor, a haptic sensor, a piezo sensor, and a vibration motor.

An intensity of the vibration of the stimulation devices 103a and 103b may be an intensity that a healthy person feels the vibration and an intensity that is difficult to feel if there is a problem with a tactile sense.

Unlike the embodiments described above, each of the stimulation devices 103a and 103b may be operated twice or more. In this case, reliability of the examination can be improved by allowing the stimulation devices 103a and 103b to generate a different type of vibration each time.

In the output steps S170 and S270, the determination value may be output by voice.

A method such as storing the examined records in the controller (not shown) and outputting the recodes to the terminal 300 when necessary may be utilized.

The predetermined number of times may be set by the controller (not shown) or the server S or may be set by being input to the terminal 300 by the user before the start value input steps S110 and S210. In this instance, the predetermined number of times may be set based on the number of the stimulation devices 103a and 103b so that as many parts of the sole of the foot as possible are examined.

Unlike the embodiments described above, the check value input steps S150 and S250 and the determination value deriving steps S160 and S260 may be omitted, and the input steps S110 and S210, the selection steps S120 and S220, and the operation steps S140 and S240 may be repeated the predetermined number of times.

Although the embodiments have been described with reference to a number of illustrative embodiments thereof, numerous other modifications and embodiments may be devised by those skilled in the art that will fall within the scope of the principles of the present disclosure. In particular, various variations and modifications are possible in the component parts and/or arrangements of the subject combination arrangement within the scope of the disclosure, the drawings and the appended claims.

### DESCRIPTION OF REFERENCE NUMERALS

| | |
|---|---|
| 100a, 100b, 200a, 200b: | 101a, 101b: Communication device |
| Stimulation assembly | |
| 103a, 103b: Stimulation device | 105a, 105b, 107a, 107b: Main body |
| 109: Accommodating portion | 300: Terminal |
| 400: Output unit | 500: Input unit |
| S: Server | |

1. A method (S100) of examining a foot ulcer comprising:
a step (S110) of inputting a start value to a terminal including a controller;
a step (S120) of randomly selecting, by the controller, one stimulation device among a plurality of stimulation devices connected to a communication device included in each of a left main body and a right main body of a shape corresponding to a shape of a sole of a foot;
a step (S130) of transmitting, by the controller, a signal to the communication device connected to the selected stimulation device;
a step (S140) of operating the selected stimulation device;
a step (S150) of inputting a check value to the terminal;
a step (S160) of deriving, by the terminal, a determination value about whether the check value matches the operated stimulation device; and
a step (S170) of outputting the determination value to the terminal.

2. The method (S100) of claim 1, wherein after the random selection step (S120) to the determination value deriving step (S160) are repeated a predetermined number of times, the determination value output step (S170) is performed, and
wherein in the determination value output step (S170), the determination values derived by the predetermined number of times are synthesized and output.

3. The method (S100) of claim 2, wherein in the operation step (S1-40), the stimulation devices generate a vibration, and the stimulation devices are operated at the same intensity of stimulation.

4. A method of examining a foot ulcer comprising:
a step (S210) of inputting a start value to a terminal;
a step (S215) of transmitting the start value to a server;
a step (S220) of randomly selecting, by the server, one stimulation device among a plurality of stimulation devices included in a main body of a shape corresponding to a shape of a sole of a foot;
a step (S230) of transmitting, by the server, a signal operating the selected stimulation device to a communication device connected to the plurality of stimulation devices;
a step (S240) of operating, by the communication device, the selected stimulation device;
a step (S250) of inputting a check value to the terminal;
a step (S255) of transmitting the check value to the server;
a step (S260) of deriving, by the server, a determination value about whether the check value matches the operated stimulation device; and
a step (S270) of transmitting, by the server, the determination value to the terminal and outputting the determination value to the terminal.

5. A foot ulcer examination device comprising:
a main body of a shape corresponding to a shape of a sole of a foot;
a plurality of stimulation devices included in the main body and spaced apart from each other; and
a communication device connected to the plurality of stimulation devices and a terminal randomly selecting the stimulation device to be operated, the communication device being configured to operate the stimulation device selected by the terminal.

6. A foot ulcer examination system comprising:
a main body of a shape corresponding to a shape of a sole of a foot;
a plurality of stimulation devices included in the main body and spaced apart from each other;
a communication device connected to the plurality of stimulation devices and configured to operate the stimulation devices; and
a terminal including:
an input unit configured to obtain an input value;
a controller connected to the input unit and the communication device, the controller being configured to perform a calculation on the input value and randomly select the stimulation device to be operated; and
an output unit configured to output an output value processed by the controller.
